(19) United States
(12) Patent Application Publication
(10) Pub. No.: US 2014/0069442 A1
(43) Pub. Date: Mar. 13, 2014

(54) METHOD OF PRINTING INDICIA ON EARPLUGS, APPARATUS FOR PERFORMING SUCH METHOD AND EARPLUGS MADE FROM SAME

(71) Applicants: Lansing M. Lewis, III, Tallahassee, FL (US); Drew E. O'Connell, SR., Cold Spring Harbor, NY (US)

(72) Inventors: Lansing M. Lewis, III, Tallahassee, FL (US); Drew E. O'Connell, SR., Cold Spring Harbor, NY (US)

(73) Assignee: Cirrus Healthcare Products, LLC, Cold Spring Harbor, NY (US)

(21) Appl. No.: 13/610,462

(22) Filed: Sep. 11, 2012

(51) Int. Cl. A61F 11/08; B41F 33/00

(52) U.S. Cl. 128/864; 101/483; 101/35

(57) ABSTRACT

An embodiment of the present invention is directed to an earplug that includes a cylindrical body having a pair of faces positioned on opposite sides of the cylindrical body, and indicia printed on at least one face of the pair of faces. Other embodiments of the present invention are directed to a method and a system for producing the earplug having indicia thereon. The method includes forming the earplug with the cylindrical body and the pair of faces, and printing the at least one indicia on at least one face of the pair of faces. The system includes an extrusion machine configured to form the earplug, a smoothing roller configured to smooth the earplug, and a printing apparatus configured to print the indicia on the earplug. The earplug is formed silicone comprised of 60-90% vinyl stopped dimethypolysiloxane, 10-30% treated fumed silica and 0.5% or less of a fluoropolymer by weight.

### BACKGROUND OF THE INVENTION

1. Field of the Invention

This invention relates to printing indicia on earplugs, and more particularly, to a method and apparatus for printing indicia on silicon earplugs, and silicon earplugs produced from the same.

2. Description of Related Art

Earplugs are generally used for sound attenuation and/or to prevent foreign particles from entering a user's ear and/or ear canal. The earplugs may be made from a variety of materials, such as foam, rubber and/or silicone, depending upon the intended use and purposes of the earplugs. However, it is desirable that the earplugs be capable of removable insertion into the user's ear and/or ear canal while fitting comfortably so as to not cause pain and/or discomfort to the user. Accordingly, many earplugs are made from such materials that are pliable and moldable in order to comfortably fit within the ear and/or ear canal of the user. However, due to the pliability of some of the materials used to form the earplugs, it may be difficult to print indicia on the earplugs. Therefore, what is needed is a process for printing indicia on pliable earplugs and an earplug that is capable of receiving printed indicia.

### SUMMARY OF THE INVENTION

The present invention is designed to overcome the above noted limitations that are attendant upon the use of conventional earplugs and, toward this end, it contemplates the provision of a novel earplug having indicia printed thereon, a method, system and apparatuses for producing such earplug.

Accordingly, it is an object of the present invention to provide an earplug that has a printing surface formed thereon for receiving printed indicia on the earplug.

It is another object of the present invention to provide an earplug that is sufficiently pliable in order to be inserted into a user's ear and/or ear canal, but sufficiently resilient so as to provide a suitable printing surface for the reception of printed indicia.

It is yet another object of the present invention to provide an earplug that has indicia printed thereon, where indicia are visible when the earplug is inserted into a user's ear and/or ear canal.

It is still another object of the present invention to provide a method and system for producing an earplug that has a printing surface formed thereon for receiving printed indicia on the earplug.

It has now been found that the foregoing and related objects can be readily attained with an exemplary embodiment of the present invention that is directed to an earplug that includes a cylindrical body having a pair of faces positioned on opposite sides of the cylindrical body, and at least one indicia printed on at least one face of the pair of faces.

In accordance with this exemplary embodiment of the present invention, the earplug may be formed from silicone that is made of 60-90% vinyl stopped dimethypolysiloxane by weight, 10-30% treated fumed silica by weight and 0.5% or less of a fluoropolymer by weight.

In accordance with this exemplary embodiment of the present invention, the fluropolymer is polytetrafluoroethylene resin.

In accordance with this exemplary embodiment of the present invention, the at least one indicia comprises a mark, logo, trademark, character, image, letter, number, symbol, design, artwork, pattern or combination thereof.

In accordance with this exemplary embodiment of the present invention, the cylindrical body of the earplug has a height between 9 to 11 millimeters, and each face of the pair of faces has a diameter between 14 to 16 millimeters.

Another exemplary embodiment of the present invention is directed to a method for producing at least one earplug having at least one indicia thereon, and the method may include forming the at least one earplug that has a cylindrical body having a pair of faces positioned on opposite sides of the cylindrical body, and printing the at least one indicia on at least one face of the pair of faces.

In accordance with this exemplary embodiment of the present invention, the at least one earplug is formed silicone comprised of 60-90% vinyl stopped dimethypolysiloxane by weight, 10-30% treated fumed silica by weight and 0.5% or less of a fluoropolymer by weight.

In accordance with this exemplary embodiment of the present invention, the fluropolymer is polytetrafluoroethylene resin.

In accordance with this exemplary embodiment of the present invention, forming the at least one earplug further includes extruding the cylindrical body of the at least one earplug from an extrusion machine, contacting the cylindrical body with a supporting surface to form a first face of the pair of faces, and cutting the cylindrical body along a cylindric section of the cylindrical body to form a second face of the pair of faces.

In accordance with this exemplary embodiment of the present invention, forming the at least one earplug further includes compressing the second face of the pair of faces with a smoothing roller.

In accordance with this exemplary embodiment of the present invention, the at least one indicia comprises a mark, logo, trademark, character, image, letter, number, symbol, design, artwork, pattern or combination thereof.

In accordance with this exemplary embodiment of the present invention, printing the at least one indicia on at least one face of the pair of faces of the earplug includes applying an ink to a printing plate, contacting the printing pad with a printing pad, and contacting the at least one face of the pair of faces of the earplug with the printing pad to print at least part of the at least one indicia on the earplug.

In accordance with this exemplary embodiment of the present invention, the ink has a composition of 10-25% cyclohexanone by weight, 2.5-10% 2-Butoxyethyl Acetate by weight, 2.5-10% 2-methoxy-1-methylethyl acetate by weight and 2.5-10% solvent naphtha by weight.

Another exemplary embodiment of the present invention is directed to a system for producing at least one earplug having at least one indicia thereon, and the system may include an extrusion machine configured to form at least one earplug having a cylindrical body with a pair of faces positioned on opposite sides of the cylindrical body, a smoothing roller configured to compress at least one face of the pair of faces of the earplug, and a printing apparatus configured to print the at least one indicia on at least one face of the pair of faces of the earplug.

In accordance with this exemplary embodiment of the present invention, the at least one earplug is formed silicone comprised of 60-90% vinyl stopped dimethypolysiloxane by weight, 10-30% treated fumed silica by weight and 0.5% or less of a fluoropolymer by weight.

In accordance with this exemplary embodiment of the present invention, the fluropolymer is polytetrafluoroethylene resin.

In accordance with this exemplary embodiment of the present invention, the at least one indicia comprises a mark, logo, trademark, character, image, letter, number, symbol, design, artwork, pattern or combination thereof.

In accordance with this exemplary embodiment of the present invention, the system may also include a fixture configured to hold the at least one earplug for printing of the at least one indicia on at least one face of the pair of faces of the earplug by the printing apparatus.

In accordance with this exemplary embodiment of the present invention, the printing apparatus is configured to apply at least one ink to the earplug in order to print the at least one indicia on the earplug.

In accordance with this exemplary embodiment of the present invention, the ink has a formulation of 10-25% cyclohexanone by weight, 2.5-10% 2-Butoxyethyl Acetate by weight, 2.5-10% 2-methoxy-1-methylethyl acetate by weight and 2.5-10% solvent naphtha by weight.

### BRIEF DESCRIPTION OF THE SEVERAL VIEWS OF THE DRAWINGS

For a fuller understanding of the nature and object of the present invention, reference should be had to the following detailed description taken in connection with the accompanying drawings, in which:

FIG. 1 is a perspective view of an exemplary earplug with indica printed thereon according to the present invention;

FIG. 2 is a perspective view of a plurality of earplugs arranged in a configuration on a supporting sheet for the printing of indicia thereon;

FIG. 3 is a top plan view of an exemplary smoothing roller that may be used in accordance with the present invention in the process of printing indicia on earplugs;

FIG. 4 is a cross-sectional view along line 4-4 in FIG. 3 of the exemplary smoothing roller;

FIG. 5 is perspective view of an exemplary fixture that may be used in accordance with the present invention in the process of printing indicia on earplugs;

FIG. 6 is a front view of the exemplary fixture from FIG. 5;

FIG. 7 is a perspective view showing how an exemplary fixture may be affixed to an exemplary conveyor and how earplugs may be positioned and retained on the exemplary fixture;

FIG. 8 is a top plan view of an exemplary printing pad that may be used in accordance with the present invention in the process of printing indicia on earplugs;

FIG. 9 is a perspective view of the exemplary printing pad of FIG. 8;

FIGS. 10A-10E shows top plan views of a printing sequence of an exemplary multi-color indicia on an earplug in according to the present invention in which one color of the multi-color indicia is printed on the earplug at a time;

FIG. 11 is a simplified view showing exemplary components of an exemplary system for printing indicia on earplugs in accordance with the present invention; and

FIG. 12 is a left side view of an exemplary silicone extrusion machine that may be used in accordance with the present invention in the process of printing indicia on earplugs.

### DETAILED DESCRIPTION OF THE INVENTION

The present invention now will be described more fully hereinafter with reference to the accompanying figures, in which exemplary embodiments of the invention are shown. The invention may, however, be embodied in many different forms and should not be construed as limited to the embodiments set forth herein. Like reference numerals refer to like elements throughout.

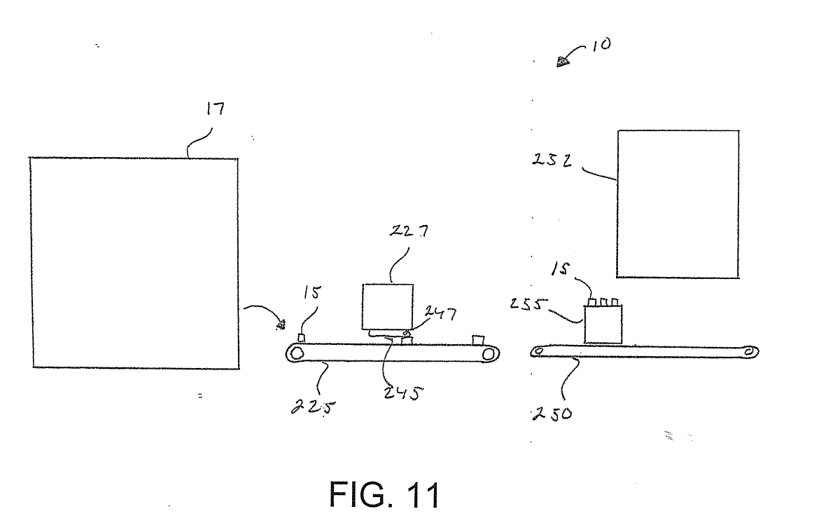

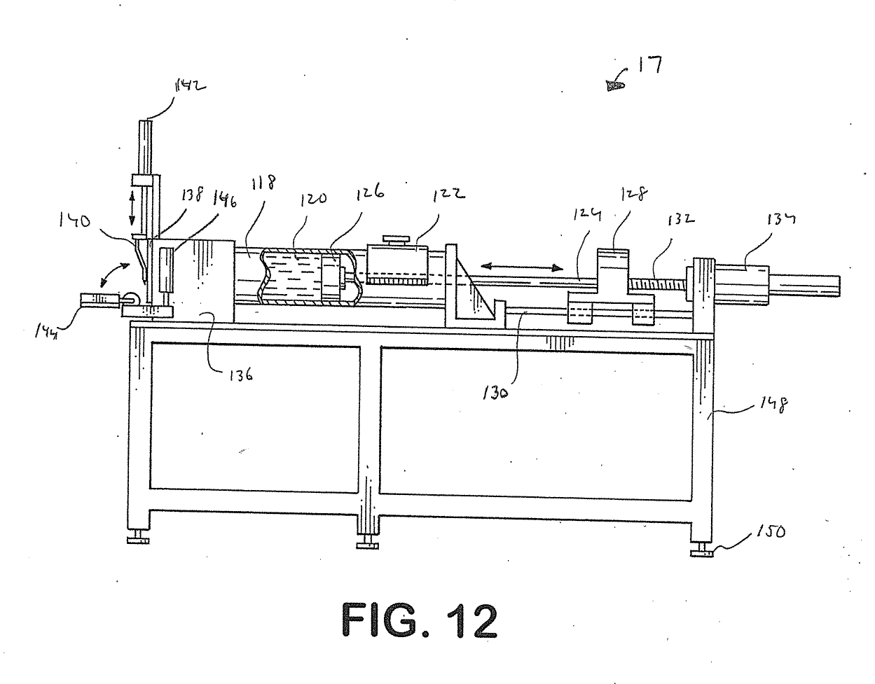

Referring now to FIG. 11, therein illustrated is an exemplary system, generally indicated by reference numeral 10, according to the present invention that may be used in the production of earplugs 15 with indicia printed thereon. The earplugs 15 may be formed from silicone, and preferably a silicone having a formulation of 60-90% vinyl stopped dimethypolysiloxane by weight, 10-30% treated fumed silica by weight and 0.5% or less of a fluoropolymer by weight, for example polytetrafluoroethylene resin. The system 10 according to the present invention may include a silicone extrusion machine 17 that is configured to extrude the earplugs 15 with the desired shape, configuration and consistency. The silicone extrusion machine 17 may be any suitable silicone extrusion machine as known to one of ordinary skill in the art, and for example may be the silicone extrusion machine, generally indicated by reference numeral 17, in FIG. 12. The silicone extrusion machine 17 may include one or more extrusion chambers 118 configured to receive and contain an amount of silicone 120. Each of the one or more extrusion chambers 118 may be formed from a cylindrical tube, and may preferably be made from stainless steel. Each of the one or more extrusion chambers 118 may contain a hatch 122 positioned so as to allow loading of the amount of silicone 120 into to extrusion chamber 118. The silicone extrusion machine 17 also includes a plunger 124 that includes a plug 126 and carriage 128. The plug 126 is mounted to one end of the plunger 124, and is dimensioned to fit snugly into the extrusion chamber 118 so as to be able to push the amount of silicone 120 towards one end of the extrusion chamber 118. The carriage 128 is mounted to the opposite end of the plunger 124 and sits on at least one guide rail 130 positioned substantially parallel to the extrusion chamber 118, preferably two guide rails 130 may be used to hole the carriage 128. The carriage 128 is operatively connected to a jack screw 132 that is operatively connected to a motor 134, for example a servo motor, configured to cause rotation of the jack screw 132, and thereby move the carriage 128 towards or away from the extrusion chamber 118 depending upon the operation of the motor 134. The movement of the carriage 128 in turn causes movement of the plunger 124, which results in movement of the plug 126 to either back off from the amount of silicone 120 or force the amount of silicone 120 out of the extrusion chamber 118. Preferably, the plunger 124 is configured to move at a speed of approximately 0.035 inches per second when the plunger 124 is moved to force the amount of silicone out of the extrusion chamber 118, and when the plunger 124 moves from a position backed off of the amount of silicone 120 to contact the amount of silicone 120 the plunger 124 is configured to move at a speed of approximately 0.166 inches per second. Each of the one or more extrusion chambers 118 is attached to a front mount 136 that includes a die plate 138 attached to the opposite side of the front mount 136 as the side of the front mount 136 the extrusion chamber 118 is attached to. The die plate 138 contains a plurality of holes (not shown), preferably approximately twelve, that are dimensioned to allow portions of the amount of silicone 120 to be extruded therefrom as a result of pressure from the plunger 124 placed upon the amount of silicone 120. The die plate 138 may be made from a suitable stainless steel. The silicone extrusion machine 17 also includes a cutting blade 140 positioned in close proximity to the die plate 138, and operative to cut a desired portion of the amount of silicone 120 that has been extruded through the die plate 138. The cutting blade 140 is operatively connected to a first piston 142 that is configured to control the movement and speed of the cutting blade 140. The silicone extrusion machine 17 may also include a flipper 144 that is positionable between a first position adjacent and substantially parallel to the die plate 138 in order to receive the portions (not shown) of the amount of silicone 120 that are extruded from the die plate 138 and cut off by the cutting blade 140. It is understood that the portions of the amount of silicone 120 may be in the form of earplugs, as discussed further below. The flipper 144 is also positionable in a second position that is substantially perpendicular to the die plate 138 and spaced away from the die plate 138. The flipper 144 may be movable between the first position and the second position by the action of a second piston 146. The silicone extrusion machine 17 may be mounted on a supporting base 148 that may include one or more leveling feet 150.

The silicone extrusion machine 17 is configured to produce substantially cylindrical earplugs 15 as shown for example in FIG. 2. The silicone extrusion machine 17 may produce a plurality of earplugs 15, for example twelve as shown in FIG. 2, during each extrusion process, and each of the earplugs 15 may be extruded onto a supporting sheet 219, which may for example be an acetate sheet. One supporting sheet 219 that is dimensioned to hold all of the earplugs 15 extruded from the silicone extrusion machine during each extrusion process may be used or multiple supporting sheets 219 may be used to hold all of the earplugs 15. For example, a single supporting sheet 219 may be used to hold all twelve earplugs 15 as shown in FIG. 2, or two (not shown) supporting sheets 219 may each hold six of the twelve earplugs 15. Preferably, the supporting sheet 219 configured to hold twelve earplugs 15 may be approximately 2.5 inches by 3.5 inches, and each supporting sheet 219 configured to hold six earplugs may be approximately 2.5 inches by 1.72 inches. However, it is understood that the present invention is not limited to the actual dimensions of the supporting sheet 219. Each of the earplugs 15 has a pair of faces 221 positioned on opposite sides of a cylindrical body 223. One of the pair of faces 221 of the earplug 15 is affixed to the supporting sheet 219, while the opposing face 221 of the earplug 15 is positioned away from and substantially parallel to the supporting sheet 219.

Referring again to FIG. 11, the system 10 may also include a means of conveyance 225 for moving the earplugs 15 from a first location to a second location of the system 10. For example, the means of conveyance 225 may be a conveyor belt or other type of conveyor. The system 10 may also include a smoothing roller 227 positioned along the means of conveyance 225 so that the earplugs 15 moving along the means of conveyance 225 are positioned to have the face 221 of the earplug 15 flattened and/or smoothed by the action of the smoothing roller 227.

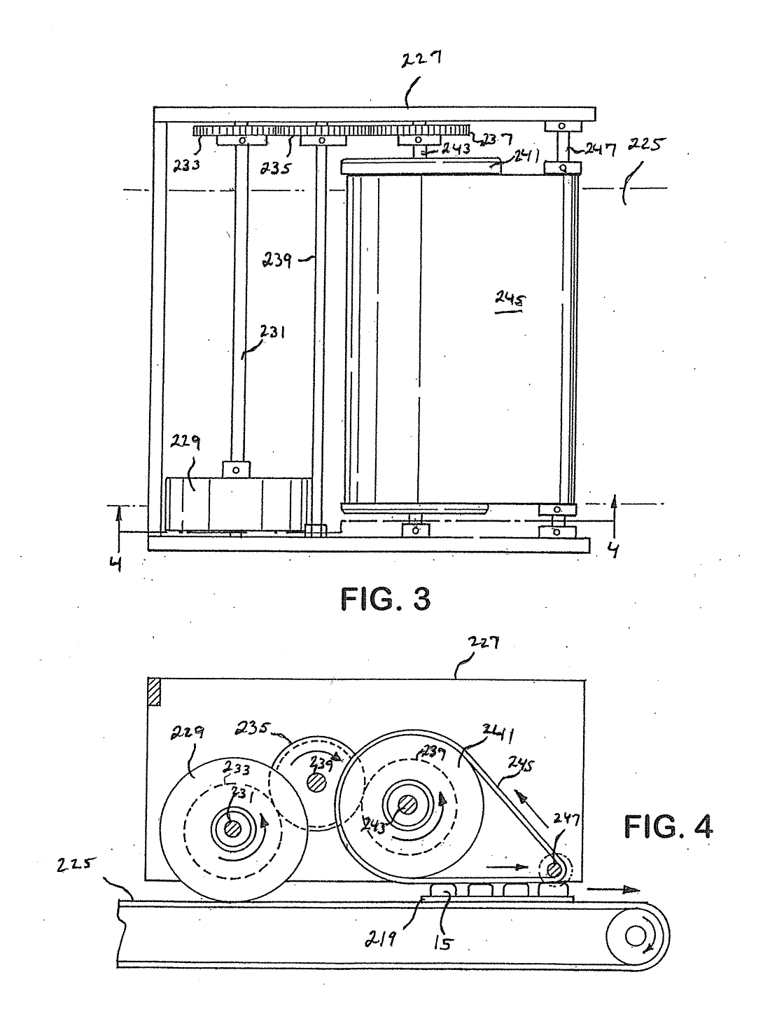

Referring now to FIGS. 3 and 4 an exemplary smoothing roller 227 that may be used in the system 10 according to the present invention is shown. The smoothing roller 227 includes a drive wheel 229 configured for rotational movement within the smoothing roller 227, and positioned in contact with the means of conveyance 225 so that movement of the means of conveyance 225 is imparted to the drive wheel 229. In this manner, the drive wheel 229 may be rotated when the means of conveyance 225 is in operation. The smoothing roller 227 also include a first drive shaft 231 that is connected to the drive wheel 229 and configured to transfer the rotational motion of the drive wheel 229 to a first gear 233 positioned on an end of the first drive shaft 231 opposite the drive wheel 229. The first gear 233 is operationally engaged with a second gear 235, and the second gear 235 is operationally engaged with a third gear 237. The second gear 235 may be positioned on a rotatable shaft 239 in order to assist in the proper orientation of the second gear 235. The purpose of the second gear 235 is to transfer the rotational motion from the first gear 233 into rotational motion in a direction opposite the rotational motion of the first gear 233. For example, as shown in FIG. 4 the first gear 233 is configured to rotate in a counter-clockwise direction as a result of the action of the means of conveyance 225 upon the drive wheel 229. This counter-clockwise rotation of the first gear 233 is transferred to clockwise rotation of the second gear 235, which is then transferred to the third gear 237 so that the third gear 237 rotates in a counter-clockwise direction. The smoothing roller 227 further includes a roller 241 operatively connected to the third gear 237 so that the rotation, in this example counter-clockwise rotation, is transferred to the roller 241. The roller 241 may be formed from a plastic tube with weight supporting end caps, and may be hard mounted to a second drive shaft 243 operatively connected to the third gear 237. It is understood that the roller 241 may be any suitable size, and preferably may be approximately three inches in diameter, but it is understood that the present invention is not limited to any particular sized roller 241. The smoothing roller 227 also includes a belt 245 operatively engaged with the roller 241. The belt 245 may be constructed from a cloth base and an adhesive backed wear-resistant slippery ultra-high molecular weight polyethylene. The adhesive side of the ultra-high molecular weight polyethylene is applied to the cloth base so that the ultra-high molecular weight polyethylene of the belt 245 is what makes contact with the earplugs 15. It is understood that any suitable ultra-high molecular weight polyethylene or other similar materials may be used to form the belt 245, and that a suitable exemplary ultra-high molecular weight polyethylene that may be used would be Part No. 76445A726 purchased from McMaster-Carr Supply Company of Robbinsville, N.J. The adhesive on the ultra-high molecular weight polyethylene may be any suitable adhesive, for example an acrylic adhesive. Furthermore, it is understood that the ultra-high molecular weight polyethylene may be any suitable thickness and width. For example, the ultra-high molecular weight polyethylene may be approximately 0.0045 to 0.0115 thick, and preferably approximately 0.0045 thick, and preferably approximately six inches wide. It is understood that the present invention is not limited to any particular thickness and/or width of the ultra-high molecular weight polyethylene and/or the belt 245 that may be used with the smoothing roller 227.

Still referring to FIGS. 3 and 4, the smoothing roller 227 also includes a free-spinning roller 247 on which the belt 245 extends around, so that the belt 245 extends around both the roller 241 and the free-spinning roller 247. The free-spinning roller 247 is positioned and configured so that the belt 245 is pulled sharply off of the earplugs 15 as the earplugs travel along the means of conveyance 225 and through the smoothing roller 227. The smoothing roller 227 may be configured so that the height of the roller 241, the belt 245 and the free-spinning roller 247 is adjustable in accordance with the desired sizes of the earplugs 15 that are intended to be smoothed by the smoothing roller 227. For example, the belt 245 of the smoothing roller may be positioned approximately nine millimeters from the means of conveyance 225. It is understood that the purpose of the smoothing roller 227 in the system 10 is to provide a smooth printing surface on one of the faces 221 of the earplugs 15 by removing any small bumps, marks and/or distortions, and to provide earplugs 15 of uniform height. It is understood that as a result of the configuration of the smoothing roller 227 relative to the means of conveyance 225, that the smoothing roller 227 will operate at substantially the same speed as the means for conveyance 225 as a result of the drive wheel 229 being engaged with the means of conveyance 225. Preferably, the means of conveyance 225, and in turn the smoothing roller 227 operate at approximately 12 to 20 feet per minute, however it is understood that any suitable operation speed may be encompassed by the present invention.

Referring again to FIG. 11, the system 10 according to the present invention may also include a conveyor 250 that is configured to transport one or more earplugs 15 through and/or to a printing apparatus 252. The one or more earplugs 15 may be disposed on a fixture 255 in order to facilitate transport of the one or more earplugs 15 through and/or to the printing apparatus 252. It is understood that the fixture 255 is configured to hold the one or more earplugs 15, for example while the one or more earplugs 15 are affixed to the supporting sheet 219, in an appropriate position and orientation so that the printing apparatus 252 may print the desired indicia on the one or more earplugs 15.

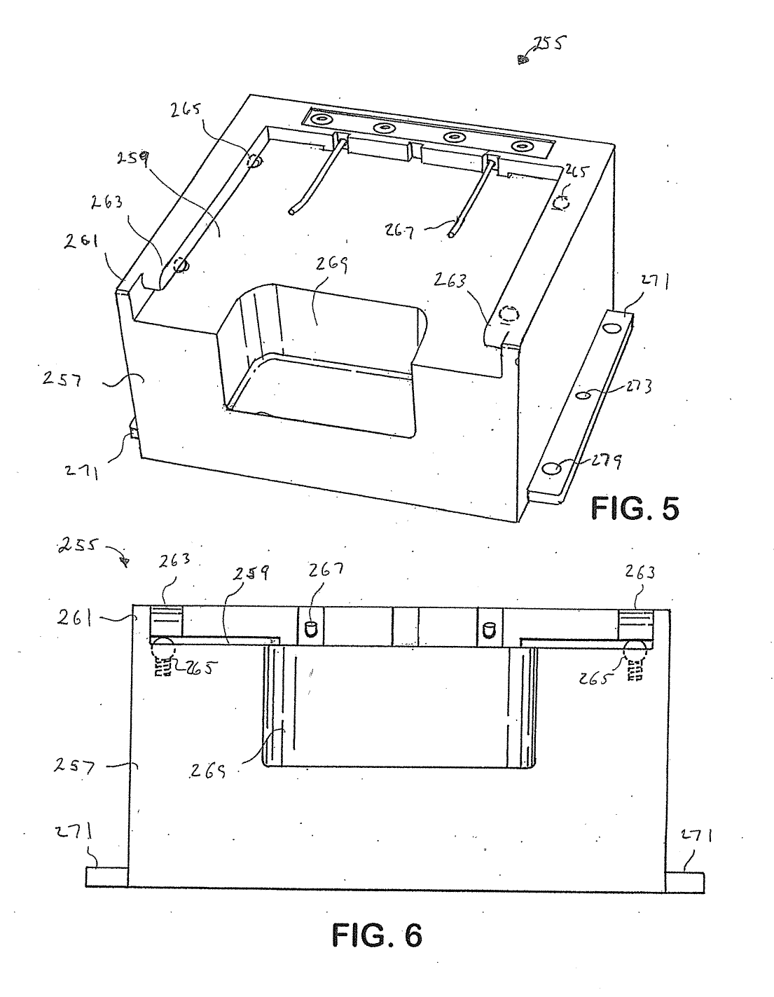

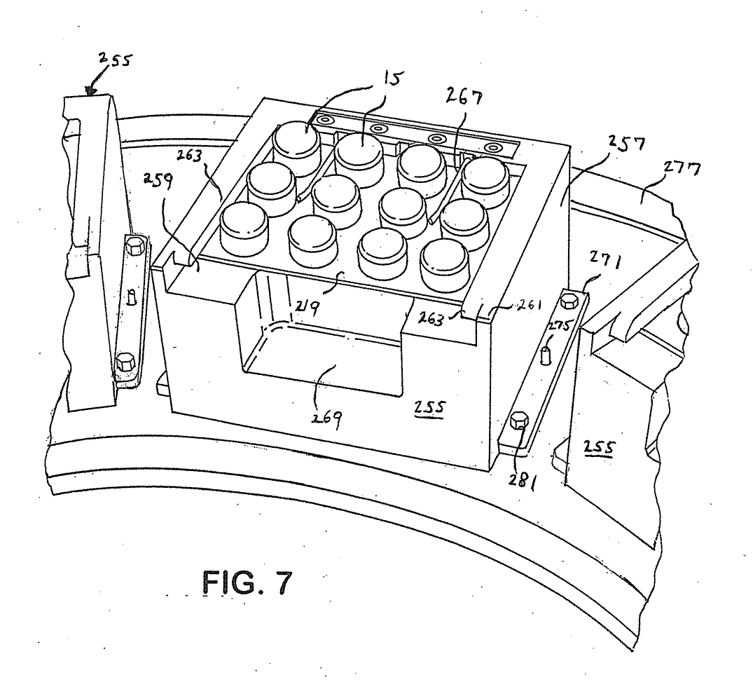

Referring now to FIGS. 5-7, the exemplary fixture 255 that may be used with the system 10 according to the present invention is therein illustrated. The fixture 255 may be formed from a generally cuboid body 257 that includes a substantially level printing shelf 259 that is configured to support at least one supporting sheet 219 containing at least one earplug 15 during the printing process. The fixture 255 may also include a raised perimeter 261 that extends around the substantially level printing shelf 259 in order to assist in the alignment and retention of the at least one supporting sheet 219 on the fixture 255. Extending from the raised perimeter 261 on at least one side of the fixture 255 is a retaining lip 263 that is spaced away from the substantially level printing shelf 259 in order to allow the at least one supporting sheet 219 to be inserted between the retaining lip 263 and the substantially level printing shelf 259. The fixture 255 may also include at least one ball plunger 265 that includes a ball and a spring that urges the ball against the retaining lip 263. The ball plunger 265 is configured so as to allow the at least one supporting sheet 219 to be inserted onto the fixture 255, and then securely retained on the fixture 255 by the action of the ball plunger 265 against the retaining lip 263. The ball plunger 265 helps to prevent and/or reduce the supporting sheet 219 from moving during the printing process. The fixture 255 may also include one or more restraint bars 267 extending from the raised perimeter 261 and positioned over the substantially level printing shelf 259. The restraint bars 267 are positioned to assist in holding the at least one supporting sheet 219 against the substantially level printing shelf 259 during the printing process. The fixture 255 may also include a recessed area 269 that facilitates insertion and/or removal of the supporting sheet 219 from the fixture 255. The fixture 255 may also include one or more extensions 271 extending from the base of the cuboid body 257, and each of the extensions 271 may include a retaining pin hole 273 designed to receive a retaining pin 275 from a conveyor, for example an elliptical conveyor 277 as shown for example in FIG. 7, in order to assist aligning the fixture 255 on the conveyor. Each of the extensions 271 may also include one or more holes 279 that are configured to receive a fastener 281, for example a screw or a bolt, that secures the fixture 255 to the conveyor.

Referring again to FIG. 11, the conveyor 250, which may for example be the elliptical conveyor 277 as shown in FIG. 7, is configured to bring the fixture 255 containing the earplugs 15 into proximity to the printing apparatus 252 so that the desired indicia may be printed thereon. The printing apparatus 252 may be any suitable printing device that is configured to print indicia on the earplugs 15. For example, the printing apparatus 252 may be an electro-pneumatic functioning pad printer produced by Pad Print Machinery of Vermont Inc. of East Dorset, Vt.. An exemplary electro-pneumatic functioning pad printer may be Model No. XP113-53S12E000 from Pad Print Machinery of Vermont Inc. that is configured to print up to five different colors on each earplug 15. It is understood that any suitable ink may be used for printing indicia on the earplugs 15 according to the present invention, but preferably the ink may have the following formulation 10-25% cyclohexanone by weight, 2.5-10% 2-Butoxyethyl Acetate by weight, 2.5-10% 2-methoxy-1-methylethyl acetate by weight and 2.5-10% solvent naphtha (petroleum) by weight. An exemplary ink that may be used in accordance with the present invention is Item No. PLT9-CMIX obtained from Pad Print Machinery of Vermont Inc. Any suitable number of dyes and or colorings may then be added to the ink in order to produce the desired colors and/or hues for the inks used in the printing of indicia on the earplugs 15.

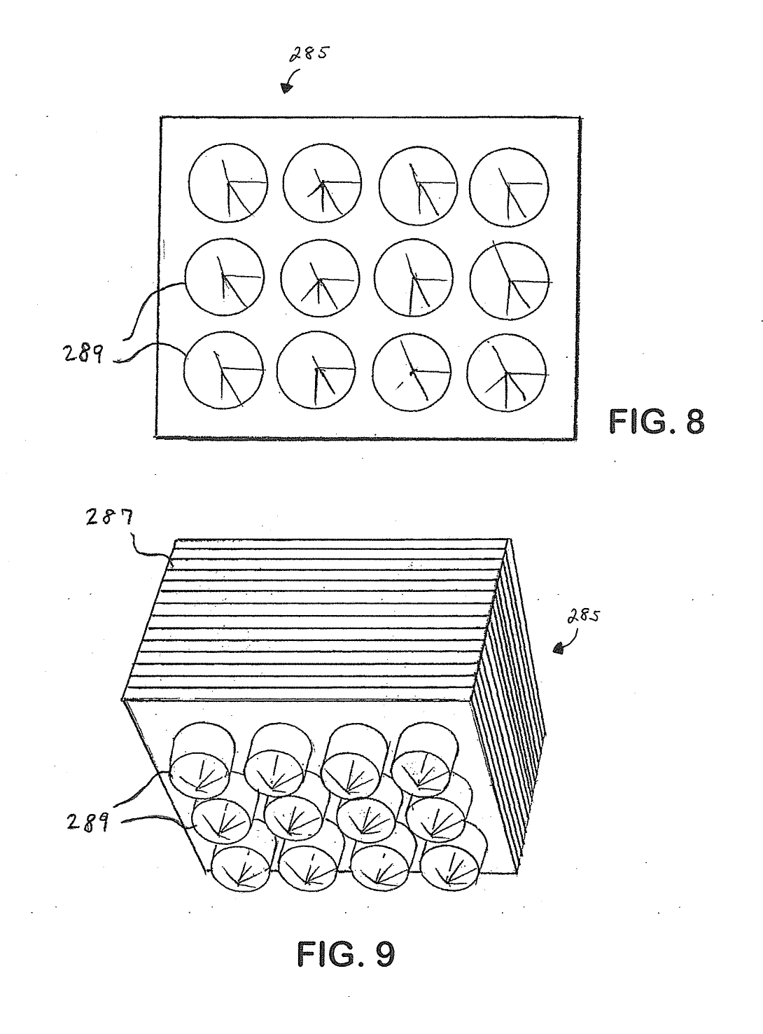

As understood by one of skill in the relevant art, during the printing process a printing pad is used to receive the ink from a printing plate that has the desired indicia formed therein. Referring now to FIGS. 8 and 9, an exemplary printing pad, generally indicated by reference numeral 285, that maybe used in accordance with the present invention is illustrated therein. The printing pad 285 includes a base portion 287 and a plurality of columnar members 289 extending from the base portion 287. The columnar members 289 are configured so as to deliver ink to the appropriate portions of the printing plate (not shown). However, it is understood that any suitable printing pads may be used and the printing pads 285 shown in FIGS. 8 and 9 are merely exemplary. Any suitable printing plates (not shown) may be used for producing the indicia on the earplugs 15 according to the present invention. The printing plates (not shown) are the vehicle that is used to hold the inked image that will be transferred by the printing pad 285 to the earplugs 15. The printing plates (not shown) are etched with the desired indicia or parts of the desired indicia for transfer onto the earplugs 15. Exemplary printing plates (not shown) that may be used for the present invention are gray alcohol wash polymer plates that may be purchased from Pad Print Machinery of Vermont Inc. However, it is understood that any suitable printing plates (not shown) that are compatible with the printing apparatus 252 used for printing indicia on the earplugs 15 may be used in accordance with the present invention.

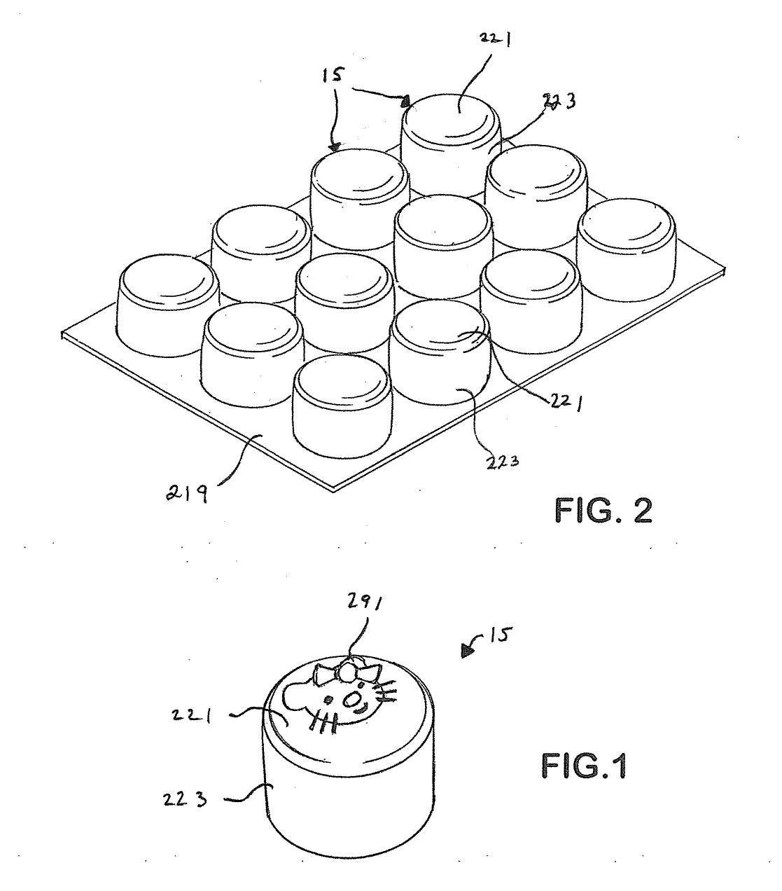

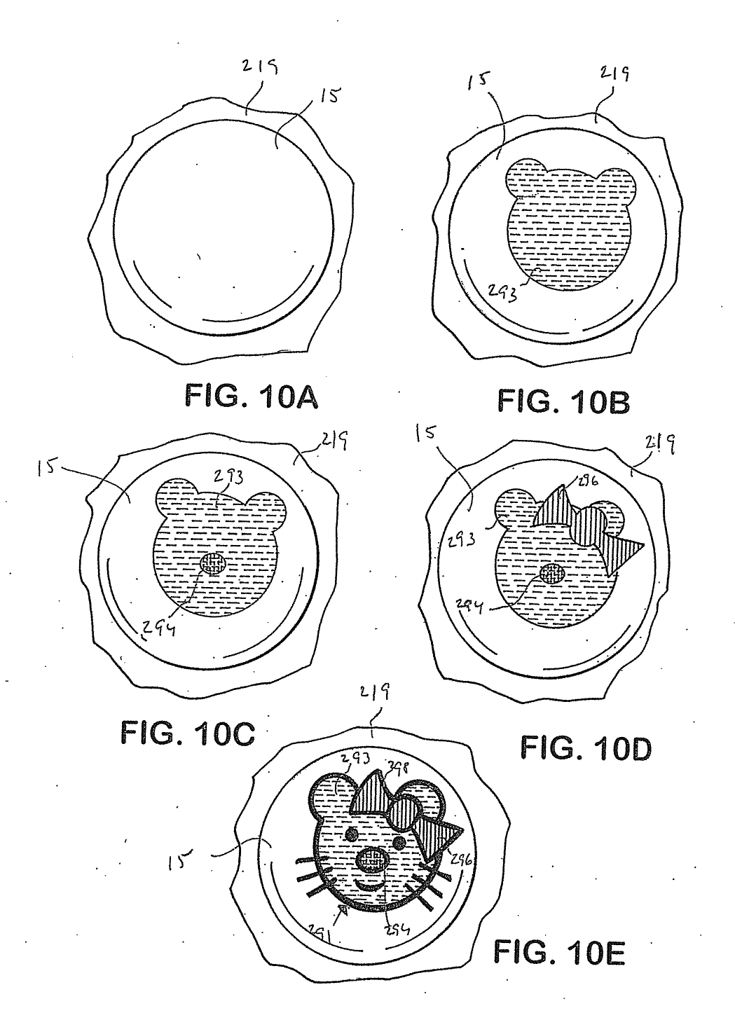

Referring now to FIGS. 1 and 10A-10E an exemplary process for printing an exemplary indicia 291 on an earplug 15 is shown. The indicia 291 that may be printed on the earplug 15 according to the present invention may be any mark, logo, trademark, character, image, letter, number, symbol, design, artwork, patterns and/or any combinations thereof. The indicia 291 may include multiple colors, and if so, may be printed on the earplug 15 in the exemplary sequence as shown in FIGS. 10A-10E. For example, FIG. 10B shows a first base color 293 printed on the earplug 15, and FIG. 10C shows a second color 294 printed on the first base color 293. Furthermore, FIG. 10D shows a third color 296 printed on the second color 294, and finally FIG. 10E shows a fourth color 298 printed on the third color 296 to form the completed indicia 291. It is understood that any suitable colors may be used for the first base color 293, the second color 294, the third color 296 and the fourth color 298. It is also understood that while printing a sequence of colors is shown in FIGS. 10A-10E in order to print the indicia 291 on the earplug 15, the present invention also contemplates printing the indicia 291 in multiple colors at one time. For example, the exemplary printing apparatus 252 mentioned above may be configured to print each color of the indicia 291 at a separate printing station (not shown) within the printing apparatus 252. In this manner, the sequence of color application of the indicia 291 shown in FIGS. 10A-10E may be conducted by the same printing apparatus 252 without having to place the earplug 15 through multiple printing apparatuses and/or change the color used in the printing apparatus 252. It is also understood that the earplug 15 itself may be any color and/or hue, whether transparent, translucent or solid, and that the color and/or hue may be produced by adding appropriate pigments and/or dyes to the silicone formulation during the production process. It is understood that the present invention is not limited to any particular color and/or hue of the earplugs 15 produced in accordance with the present invention.

The use of the exemplary earplug 15 with indicia 291 printed thereon will now be discussed with reference to FIG. 1. In use, the earplug 15 may be inserted into an ear canal (not shown) and/or outer ear (not shown) of a user (not shown) so that the indicia 291 faces away from the ear canal of the user. In this manner, the indicia 291 is visible while the earplug 15 is in use by the user. In order to insert the earplug 15 into the ear canal and/or outer ear the earplug 15, which may be made from a pliable silicone, preferably a silicone having a formulation of 60-90% vinyl stopped dimethypolysiloxane by weight, 10-30% treated fumed silica by weight and 0.5% or less of a fluoropolymer by weight, for example polytetrafluoroethylene resin, is formed by the user by applying pressure to the cylindrical body 223 of the earplug 15 in order to configure the earplug 15 into the appropriate shape and dimension to be inserted into the ear canal and/or outer ear.

The operation and use of the system 10 according to an exemplary embodiment to the present invention in producing earplugs with indicia printed thereon will now be discussed with reference to FIGS. 1-2, 5-6 and 11-12. In order to produce earplugs with indicia printed thereon, first the plunger 124 and plug 126 are moved in the extrusion chamber 118 of the silicone extrusion machine 17 so that these components are positioned towards the motor 134 and past the hatch 122 in order to permit loading of the amount of silicone 120 into the extrusion chamber 118. The amount of silicone 120 is preferably a pliable silicone, and even more preferably a silicone having a formulation of 60-90% vinyl stopped dimethypolysiloxane by weight, 10-30% treated fumed silica by weight and 0.5% or less of a fluoropolymer by weight, for example polytetrafluoroethylene resin. Next the motor 134 is activated in order to cause the plunger 124 to extrude a portion (not shown) of the amount of silicone 120 out of the die plate 138 in order to form at least one earplug 15 in the appropriate shape and configuration. Preferably, the plunger 124 is configured to move at a speed of approximately 0.035 inches per second during the extrusion of the portion of the amount of silicone 120 out of the die plate 138. The motor 134 may be configured to control the amount of travel of the plunger 124 so that an appropriately sized earplug 15 may be extruded. For example, preferably the plunger 124 may be configured to move approximately 0.07 inches during the extrusion. Prior to extrusion of one or more earplugs 15 the flipper 144 is moved into the position parallel to the die plate 138 in order to receive the one or more earplugs 15 being extruded from the extrusion machine 17. One or more supporting sheets 219 may be loaded into the flipper 144 in order to receive the one or more earplugs 15. Once the an appropriate portion (not shown) of the amount of silicone 120 is extruded, the first piston 142 forces the cutting blade 140 along the die plate 138 in order cut the one or more extruded earplugs 15 from the amount of silicone 120 still remaining the in extrusion chamber 118. Preferably, the cutting blade 140 is configured to move approximately 3 inches each half a second in order to smoothly cut the one or more extruded earplugs 15 from the extrusion machine. Once the one or more earplugs 15 have been cut by the cutting blade 140 the plunger 124 moves into a back-off position away from the amount of silicone 120 through the action of the motor 134 in order to relieve pressure on the amount of silicone 120 and prevent unwanted extrusion of the amount of silicone 120 out of the die plate 138. The flipper 144 then moves into the position perpendicular to the die plate 138 in order to permit removal of the one or more supporting sheet 219 from the flipper 144. The one or more supporting sheet 219 with earplugs 15 disposed thereon, is then moved to the means of conveyance 225 so that one of the faces 221 of each of the earplugs 15 may be acted upon by the smoothing roller 227, and thereby smoothed by the smoothing roller 227. The earplugs 15 are then moved to the fixture 255 for transport on the conveyor 250 to the printing apparatus 252. The printing apparatus 252 then applies, either in one application or a sequence of applications, the desired indicia 291 onto one of the faces 221 of the earplugs 15. The printing apparatus 252 may perform the application of the desired indicia 291 onto one of the faces 221 of the earplugs 15 by applying a suitable ink to a printing plate (not shown) that has at least a portion of the indicia 291 formed thereon, then contacting the printing plate to the printing pad 285, and then contacting the printing pad 285 to at least one of the earplugs 15.

It will thus be seen that the objects set forth above, among those made apparent from the preceding description, are efficiently attained and, since certain changes may be made in the above article without departing from the scope of this invention, it is intended that all matter contained in this disclosure or shown in the accompanying drawings, shall be interpreted, as illustrative and not in a limiting sense.

It is to be understood that all of the present figures, and the accompanying narrative discussions of corresponding embodiments, do not purport to be completely rigorous treatments of the invention under consideration. It is to be understood that the above-described arrangements are only illustrative of the application of the principles of the present invention. Numerous modifications and alternative arrangements may be devised by those skilled in the art without departing from the scope of the present invention.

1. An earplug, comprising:
a cylindrical body having a pair of faces positioned on opposite sides of the cylindrical body; and
at least one indicia printed on at least one face of the pair of faces;
wherein the earplug is formed from silicone comprised of 60-90% vinyl stopped dimethypolysiloxane, 10-30% treated fumed silica and 0.5% or less of a fluoropolymer.

2. The earplug according to claim 1, wherein the fluropolymer is polytetrafluoroethylene resin.

3. The earplug according to claim 1, wherein the at least one indicia comprises a mark, logo, trademark, character, image, letter, number, symbol, design, artwork, pattern or combination thereof.

4. The earplug according to claim 1, wherein the cylindrical body of the earplug has a height between 9 to 11 millimeters, and each face of the pair of faces has a diameter between 14 to 16 millimeters.

5. A method for producing at least one earplug having at least one indicia thereon, comprising:
forming the at least one earplug comprising a cylindrical body having a pair of faces positioned on opposite sides of the cylindrical body, and
printing the at least one indicia on at least one face of the pair of faces;
wherein the at least one earplug is formed silicone comprised of 60-90% vinyl stopped dimethypolysiloxane, 10-30% treated fumed silica and 0.5% or less of a fluoropolymer.

6. The method according to claim 5, wherein the fluropolymer is polytetrafluoroethylene resin.

7. The method according to claim 5, wherein forming the at least one earplug comprises:
extruding the cylindrical body of the at least one earplug from an extrusion machine,
contacting the cylindrical body with a supporting surface to form a first face of the pair of faces, and
cutting the cylindrical body along a cylindric section of the cylindrical body to form a second face of the pair of faces.

8. The method according to 7, wherein forming the at least one earplug further comprises compressing the second face of the pair of faces with a smoothing roller.

9. The method according to claim 5, wherein the at least one indicia comprises a mark, logo, trademark, character, image, letter, number, symbol, design, artwork, pattern or combination thereof.

10. The method according to claim 5, wherein printing the at least one indicia on at least one face of the pair of faces of the earplug comprises:
applying an ink to a printing pad,
contacting the printing pad with a printing plate, and
contacting the at least one face of the pair of faces of the earplug with the printing plate to print at least part of the at least one indicia on the earplug,
wherein the ink comprises 10-25% cyclohexanone, 2.5-10% 2-Butoxyethyl Acetate, 2.5-10% 2-methoxy-1-methylethyl acetate and 2.5-10% solvent naphtha.

11. A system for producing at least one earplug having at least one indicia thereon, comprising:
an extrusion machine configured to form at least one earplug comprising a cylindrical body having a pair of faces positioned on opposite sides of the cylindrical body;
a smoothing roller configured to compress at least one face of the pair of faces of the earplug; and
a printing apparatus configured to print the at least one indicia on at least one face of the pair of faces of the earplug;
wherein the at least one earplug is formed silicone comprised of 60-90% vinyl stopped dimethypolysiloxane, 10-30% treated fumed silica and 0.5% or less of a fluoropolymer.

12. The system according to claim 11, wherein the fluropolymer is polytetrafluoroethylene resin.

13. The system according to claim 11, wherein the at least one indicia comprises a mark, logo, trademark, character, image, letter, number, symbol, design, artwork, pattern or combination thereof.

14. The system according to claim 11, wherein the system further comprises a fixture configured to hold the at least one earplug for printing of the at least one indicia on at least one face of the pair of faces of the earplug by the printing apparatus.

15. The system according to claim 11, wherein the printing apparatus is configured to apply at least one ink to the earplug in order to print the at least one indicia on the earplug; and
wherein the ink comprises 10-25% cyclohexanone, 2.5-10% 2-Butoxyethyl Acetate, 2.5-10% 2-methoxy-1-methylethyl acetate and 2.5-10% solvent naphtha.

16. The earplug of claim 1, wherein the silicone comprised of 60-90% vinyl stopped dimethypolysiloxane, 10-30% treated fumed silica and 0.5% or less of a fluoropolymer forming the earplug is pliable.

17. The method of claim 5, wherein the silicone comprised of 60-90% vinyl stopped dimethypolysiloxane, 10-30% treated fumed silica and 0.5% or less of a fluoropolymer forming the earplug is pliable.
